(19) United States
(12) Patent Application Publication
(10) Pub. No.: US 2026/0317699 A1
(43) Pub. Date: Oct. 15, 2026

(54) FLUIDIC PROCESS AND DEVICES FOR CREATION OF FIBRILLAR PROTEIN SUPPORTS

(71) Applicant: The Regents of The University of Michigan, Ann Arbor, MI (US)

(72) Inventors: Do Hoon KIM, Ann Arbor, MI (US); Dylan B. NEALE, Somerville, MA (US); Malini MUKHERJI, Cambridge, MA (US); Joerg LAHANN, Ann Arbor, MI (US); Seongjun MUN, Ann Arbor, MI (US)

(73) Assignee: The Regents of The University of Michigan, Ann Arbor, MI (US)

(21) Appl. No.: 19/485,200

(22) PCT Filed: May 17, 2024
(86) PCT No.: PCT/US2024/029974

§ 371 Date: Nov. 17, 2025

Related U.S. Application Data
(60) Provisional application No. 63/467,443, filed on May 18, 2023.

(51) Int. Cl. C12N 5/00; C12M 1/12

(52) U.S. Cl. CPC C12N 5/0068; C12M 25/14; C12N 5/0062; C12N 2533/52

(57) ABSTRACT

Methods of making cellular support systems may include passing a bubble comprising gas through a liquid comprising an extracellular matrix protein (e.g., fibronectin) contained in a fluidic device. The bubble is passed over a target within the fluidic device to organize the extracellular matrix protein and form a deposited protein construct comprising a plurality of extracellular matrix protein fibrils on or near the target. The deposited protein construct is capable of supporting cells and promotes three-dimensional cellular growth. Fluidic devices for forming a deposited protein construct comprising a plurality of extracellular matrix protein fibrils are also provided.

### CROSS-REFERENCE TO RELATED APPLICATIONS

This application claims the benefit of U.S. Provisional Application No. 63/467,443, filed on May 18, 2023. The entire disclosure of the above-mentioned application is incorporated herein by reference.

### FIELD

The present disclosure relates to hierarchically structured protein material for three-dimensional cellular support systems and methods for making such cellular support systems.

### BACKGROUND

This section provides background information related to the present disclosure which is not necessarily prior art.

In vivo, nearly all tissue cells are found in an extracellular matrix that comprises a complex three-dimensional fibrous mesh with a distribution of fibers and voids (including various extracellular proteins) that enable complex biochemical and physical signaling by cells. Because the local microstructure plays a pivotal role for many biological functions, a wide range of methods have been developed to design precisely engineered substrates for both fundamental biological studies and biotechnological applications. However, despite the importance of multidimensional scaffolding and patterning for biological applications, their realization, especially with biomedically relevant polymers, remains challenging.

A cellular support scaffold system that includes one or more biocompatible materials and a suspended protein, such as an extracellular matrix protein, that facilitates robust cell growth and proliferation in three-dimensions, while providing control over composition and morphology is important. For example, such a cellular support scaffold system that includes one or more biocompatible materials and a suspended protein may be formed by positioning and securing a scaffold structure at a two-phase interface where there is air and a solution comprising the protein to be deposited. After positioning, the entire system including the scaffold structure may be subjected to dynamic conditions in order to induce laminar flow of the protein solution/air interface over the scaffold, for example, by mounting to a rotisserie rotator, orbital shaker, or the like. In this manner, the scaffold structure and its voids/pores act serve as a nucleation site for the protein to form suspended protein supports or bridges within the scaffold structure.

However, it would be desirable to develop new techniques for forming protein constructs suspended across two or more surfaces with specificity of region, scale, regioselectivity and/or fibrillar alignment. Moreover, it would be desirable to develop methods where the process is not limited to organizing a protein, like fibronectin, on top and across a support structure (polymer scaffolds), but rather providing the ability to organize fibronectin as a “free-standing film,” or a suspended film with no support structure.

### SUMMARY

This section provides a general summary of the disclosure, and is not a comprehensive disclosure of its full scope or all of its features.

In various aspects, the present disclosure provides a method of making a cellular support system. The method may comprise passing a bubble comprising gas through a liquid comprising an extracellular matrix protein contained in a fluidic device. The bubble is passed over a target within the fluidic device to organize the extracellular matrix protein and form a deposited protein construct comprising a plurality of extracellular matrix protein fibrils on or near the target. The deposited protein construct is capable of supporting cells and promotes three-dimensional cellular growth.

In one aspect, the extracellular matrix protein is selected from the group consisting of: fibronectins, laminins, collagens, tenascins, elastin, vitronectin, periostin, and combinations thereof.

In one aspect, the extracellular matrix protein comprises fibronectin.

In one aspect, the fluidic device has a total volume and a volume fraction of the liquid to the total volume is greater than or equal to about 0.55.

In one aspect, the plurality of extracellular matrix protein fibrils has an anisotropic orientation.

In one aspect, the target comprises a three-dimensional scaffold structure comprising at least one void and the deposited protein construct spans over the at least one void.

In one further aspect, the three-dimensional scaffold structure is formed from a material selected from the group consisting of: a metal material, a polymeric material, a composite material, a ceramic material, a biologically derived material, and combinations thereof.

In one further aspect, the three-dimensional scaffold structure comprises a polymer and is formed from a polymeric precursor or is a polymer selected from the group consisting of: polylactic acid, polyglycolide, polycaprolactone, poly(lactide-co-glycolide), poly(lactide-co-caprolactone), polyethylene glycol, starches, polydimethylsiloxane, polyurethanes, polyolefins, polyamides, celluloses, lignins, biodegradable polyesters, polystyrene, and combinations thereof.

In one aspect, the target comprises at least one surface in the fluidic device such that the deposited protein construct coats the at least one surface.

In one aspect, the target comprises a first support and a second support, so that the passing of the bubble occurs from the first support to the second support and the deposited protein construct spans from the first support to the second support.

In one further aspect, the protein construct is removed from the first support and the second support for form a free-standing film.

In one aspect, the passing the bubble comprises moving the fluidic device. The moving is selected from the group consisting of: tipping, orbital rotation, tumbling rotation, spinning, vibration, shaking, and combinations thereof.

In one aspect, the gas comprises air.

The method of claim 1, wherein the passing occurs for greater than or equal to about 5 minutes to less than or equal to about 96 hours.

In one aspect, the liquid comprising extracellular matrix protein is a protein solution having a concentration of protein from about 0.01 mg/mL to about 2.5 mg/mL.

In one aspect, the deposited protein construct occupies a surface area of greater than or equal to about 3.3 cm².

In one aspect, the deposited protein construct occupies a surface area of greater than or equal to about 9.6 cm².

In one aspect, the deposited protein construct has a dimension of greater than or equal to about 15 mm.

In one aspect, the deposited protein construct has a dimension of greater than or equal to about 35 mm.

In various aspects, the present disclosure provides a fluidic device for forming a deposited protein construct. The fluidic device may comprise a fluid-tight assembly comprising at least a first component and a second component that are assembled together to define an internal volume configured to contain a liquid comprising an extracellular matrix protein and a gas. At least one of the first component or the second component comprises at least one target on which the extracellular matrix protein will organize and form a deposited extracellular matrix protein construct comprising a plurality of extracellular matrix protein fibrils on or near the at least one target. The at least one target comprises: at least one pair of posts or at least one holding component configured to receive a three-dimensional scaffold support.

In one aspect, the internal volume defines at least one elongated channel having a cross-sectional shape selected from the group consisting of: a rectangle, a square, a circle, an oval, and combinations thereof.

In one aspect, the first component defines a first half of the at least one elongated channel and the second component defines a second half of the at least one elongated channel.

In one aspect, the first component defines the at least one elongated channel and the second component defines a planar surface.

In one aspect, the fluidic device further comprises at least one pair of recessed regions adjacent to the at least one elongated channel configured to receive the at least one holding component configured to receive the three-dimensional scaffold support.

In one aspect, the internal volume defines a plurality of distinct channels or chambers each respectively comprising a distinct target.

In one aspect, the first component defines at least one post and the second component defines at least one aperture that receives the at least one post.

In various aspects, the present disclosure also provides a cellular support comprising a deposited protein construct comprising a plurality of extracellular matrix protein fibrils aligned in an anisotropic orientation. The deposited protein construct is capable of supporting cells and promotes three-dimensional cellular growth.

In one aspect, the extracellular matrix protein is selected from the group consisting of: fibronectins, laminins, collagens, tenascins, elastin, vitronectin, periostin, and combinations thereof.

In one aspect, the extracellular matrix protein comprises fibronectin.

In one aspect, the cellular support further comprises a three-dimensional scaffold structure comprising at least one void and the deposited protein construct spans over the at least one void.

In one further aspect, the three-dimensional scaffold structure is formed from a material selected from the group consisting of: a metal material, a polymeric material, a composite material, a ceramic material, a biologically derived material, and combinations thereof.

In one further aspect, the three-dimensional scaffold structure comprises a polymer and is formed from a polymeric precursor or is a polymer selected from the group consisting of: polylactic acid, polyglycolide, polycaprolactone, poly(lactide-co-glycolide), poly(lactide-co-caprolactone), polyethylene glycol, starches, polydimethylsiloxane, polyurethanes, polyolefins, polyamides, celluloses, lignins, biodegradable polyesters, polystyrene, and combinations thereof.

In various aspects, the present disclosure further provides a method of expanding three-dimensional growth of cells. The method may comprise introducing cells to a cellular support comprising a deposited protein construct comprising a plurality of extracellular matrix protein fibrils aligned in an anisotropic orientation. The method further comprises growing the cells on the deposited protein construct in three-dimensions.

In one aspect, the cells are cancer cells.

In one aspect, the method is used for an expansion of cancer cells for the construction of cancer immunotherapies.

In one aspect, the cells are cell mixtures obtained from human tumors.

In one aspect, the cells are combinations of cells obtained from human tumors and support cells.

In one aspect, the cells are stem cells.

In one aspect, the cells are induced pluripotent stem cells.

In one aspect, the cells are adult cells selected from a group consisting of: cardiomyocytes, neurons, fibroblasts, and combinations thereof.

In one aspect, the method is used for construction of autologous whole-tumor cell immunotherapies.

In one aspect, the method is used for construction of chimeric antibody receptor (CAR) T-cell therapies.

In one aspect, the method is used for construction of organoid structures.

In one aspect, at least one cell is genetically modified during or after expansion on the cellular support.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1B show a comparison of two protein deposition processes. In FIG. 1A, an example of a meniscus flow process is shown in a cross-sectional view of a tube comprising a liquid including a protein and a fluid (e.g., air) where a meniscus is employed as a two-phase interface to deposit the protein on a target structure. In FIG. 1B, an example of a bubble flow process in accordance with certain aspects of the present disclosure is shown in a cross-sectional view of a tube comprising a liquid including a protein and a fluid (e.g., air) where an enclosed bubble is employed as a two-phase interface to deposit the protein along a target.

FIGS. 2A-2D show a fibronectin construct comprising a plurality of aligned and anisotropic fibers/fibrils deposited via a bubble flow method according to certain aspects of the present disclosure. FIG. 2A is a scanning electron microscope image showing a highly aligned and anisotropic fibronectin matrix, while a schematic drawing in FIG. 2B shows the alignment of fibers. FIG. 2C shows an average direction or alignment of the major longitudinal direction of the fibronectin fibrils. FIG. 2D shows directionality of histograms for the deposited fibronectin matrix fibrils.

FIGS. 3A-3D show a fibronectin construct comprising a plurality of misaligned fibers/fibrils deposited via a meniscus flow method of deposition. FIG. 3A shows a scanning electron microscope image of the fibronectin matrix. FIG. 3B shows a representative schematic drawing of the deposited fibers lacking alignment or anisotropy. FIG. 3C shows an average direction or alignment of the major longitudinal direction. FIG. 3D shows directionality of histograms for the deposited fibronectin matrix fibrils via the meniscus method.

FIGS. 4A-4C show an example of a system in which a bubble flow method of making a cellular support system may be conducted in accordance with certain aspects of the present disclosure. FIG. 4A shows the system, while FIGS. 4B and 4C show two respective variations of microfluidic devices. In FIG. 4B, a first microfluidic device includes a target region in the form of a plurality of support posts across which a protein construct comprising a plurality of extracellular matrix protein fibrils may be deposited. In FIG. 4C, a second microfluidic device includes a target region with a three-dimensional polymeric scaffold support having voids in a protein construct comprising a plurality of extracellular matrix protein fibrils may be deposited.

FIGS. 5A-5C show a fluidic (e.g., a microfluidic) device having a holding component that may receive one or more three-dimensional polymeric scaffolds in accordance with certain aspects of the present disclosure. FIG. 5A shows a side view of the fluidic device, FIG. 5B shows a top view of the fluidic device, and FIG. 5C shows a top view of the device having a liquid and gas disposed therein.

FIG. 6 shows a fluidic device having a tumbling design similar to that described in the context of FIGS. 5A-5C being rotated or tumbled to coat three-dimensional scaffolds in accordance with certain aspects of the present disclosure.

FIG. 7 shows yet another fluidic (e.g., a microfluidic) device having a tumbling design prepared in accordance with certain other aspects of the present disclosure.

FIGS. 8A-8C show different variations of fluidic devices having a tumbling design prepared in accordance with certain aspects of the present disclosure. FIG. 8A shows a microfluidic device having a two-part design, where one component has a plurality of full rectangular channels formed therein. FIGS. 8B-8C show a microfluidic device having a two-part design, where a first component has a plurality of half-channels formed therein and a second component has a plurality of half-channels formed therein, which when assembled form a full rectangular channel.

FIG. 9 shows another variation of a two-part fluidic device having a tumbling design where a first component has a half-channel formed therein and a second component has a half-channel formed therein, which when assembled forms a full cylindrical channel prepared in accordance with certain aspects of the present disclosure.

FIGS. 10A-10B show yet another variation of a fluidic (e.g., a microfluidic) device having a two-part tumbling design where an assembly comprises a plurality of cylindrical channels prepared in accordance with certain other aspects of the present disclosure.

FIGS. 11A-11B show yet another variation of a fluidic (e.g., a microfluidic) device having a two-part tumbling design where an assembly comprises a plurality of rectangular channels prepared in accordance with certain other aspects of the present disclosure.

FIG. 12 shows a mold forming a fluidic device in accordance with certain aspects of the present disclosure.

FIG. 13 shows yet another variation of a fluidic (e.g., a microfluidic) device having a three-part design where an assembly comprises a plurality of round platforms with posts for forming protein constructs prepared in accordance with certain other aspects of the present disclosure.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

In certain aspects, the present disclosure provides new methods and devices to induce create a fibrillar matrix of a protein, such as an extracellular matrix protein, with the ability to manipulate fluid shearing dynamics, such that the following can be achieved by way of non-limiting example: controlled matrix anisotropy, control over scale (micron to centimeter), and control over the location of deposition to enable complex cell culture arrays.

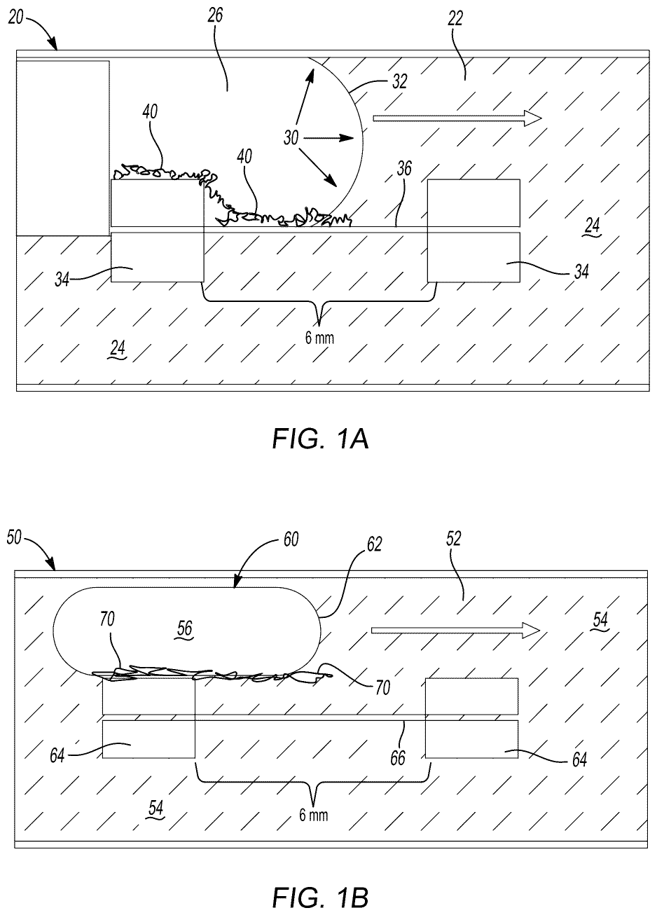

FIGS. 1A and 1B show a comparison of two protein deposition processes, more specifically two methods of depositing proteins to form a plurality of fibrils. A protein as used herein is a polypeptide chain comprising bonded amino acids, where the polypeptide chain has undergone folding (including primary, secondary, and tertiary folding) to form the complex folded molecule. In certain aspects, the protein has greater than 50 amino acids. The protein is initially dissolved as a solubilized protein in a liquid. After conducting the methods described herein, the dissolved protein is transformed into an insoluble protein construct, notably without the need for use of cells to do so. In certain aspects, the protein may be an extracellular protein and may be one or more extracellular matrix proteins. An extracellular matrix protein is one or more of the large structural fibrillar proteins often found physiologically in the extracellular matrix (ECM) of animals or plants. For example, the extracellular matrix proteins may include one or more proteins selected from the group consisting of: fibronectins, laminins, collagens, tenascins, elastin, vitronectin, periostin, and combinations thereof. Such extracellular matrix proteins can form suspended structures, whether in the form of suspended bridges or suspended free-standing films. In certain variations, the one or more extracellular proteins are selected from the group consisting of: fibronectins, laminins, collagens, and combinations thereof. In yet other variations, the one or more extracellular proteins are selected from the group consisting of: fibronectins, laminins, and combinations thereof. In further variations, the one or more extracellular proteins comprises fibronectins. Proteins that do not form suspended protein supports can be combined with/co-assembled with proteins that do form suspensions to create deposited constructs containing multiple proteins. Thus, the composition forming the protein construct may comprise a plurality of distinct proteins, including one of the aforementioned extracellular matrix proteins.

The deposited protein constructs described herein can include mixtures of such proteins, each at different relative ratios. In certain variations, a ratio of fibronectin to laminin in a protein mixture may be at a ratio of about 9:1. In other variations, a ratio of fibronectin to tenascin c may be at a ratio of about 19:1. Either of the protein deposition methods described above can form a deposited protein construct comprising a plurality of extracellular matrix protein fibrils, where shearing at a liquid-gas interface draws insoluble fibrils (e.g., fibronectin fibrils) out from the solution (e.g., a network of fibronectin, such as globular fibronectin, that forms at the fluid interface).

By fibril, it is meant that fibers or fibrous structures are formed that have an evident longitudinal axis, which is longer than the other dimensions (e.g., diameter or width), thus having an axial geometry. Generally, an aspect ratio (AR) for cylindrical or fibrous shapes is defined as AR=L/D, where L is the length of the longest axis (here the major longitudinal axis) and D is the diameter of the fiber. Suitable fibers formed in accordance with the present technology generally have high aspect ratios, for example, ranging from at least about 100 to in excess of 1,000, for example, optionally in excess of about 5,000 or more and in certain variations 10,000 or more. The protein construct that is formed is capable of spanning void regions, whether one or more voids in a support scaffold or a distance between supports to form a more extended bridge-like structure that may be later removed from the supports to form a free-standing film where the protein construct having the plurality of extracellular matrix protein fibrils can support an additional weight of cells that adhere to it.

In FIG. 1A, an example of a meniscus flow process is shown in a cross-sectional view of a tube comprising a liquid including a protein and a fluid (e.g., air) where a meniscus is employed as a two-phase interface to deposit the protein on a target structure. Notably, the solid surfaces are a third phase in the system. In FIG. 1B, an example of a bubble flow process in accordance with certain aspects of the present disclosure is shown in a cross-sectional view of a tube comprising a liquid including a protein and a fluid (e.g., air) where an enclosed bubble is employed as a two-phase interface to deposit the protein along a target (which may be a solid third phase).

As shown in FIG. 1A, a first fluidic device 20 may be a tube, a cylinder, or other container that includes an interior region 22 that may be sealed and fluid-tight and contains both a liquid 24 and gas (e.g., air) 26. The gas 26 defines a meniscus 30 at a two-phase interface 32 between the liquid 24 and gas 26. Two supports 34 are disposed in the interior region 22 that support a porous polymer structure 36 or three-dimensional scaffold structure. By way of non-limiting example, as shown, a distance between the two supports 34 is about 6 mm. The arrow shows a direction that the meniscus 30 advances through the interior region 22 of the fluidic device 20. As the meniscus 30 advances past the first of the supports 34, it deposits a protein construct 40 that comprises a fibrillar matrix comprising the plurality of extracellular matrix protein fibrils. By shearing the two-phase interface 32 over the target surfaces (e.g., supports 34 and porous polymer structure 36), fibrils, such as insoluble fibronectin fibrils, are drawn out from the liquid comprising the protein (e.g., from a network of fibronectin, which is not in a fibrillar form but can be assumed to be in globular state that forms at the fluid interface 32). The porous polymer structure 36 may be secured where the gas-liquid solution interface 32 passes, as shown in FIG. 1A. In the meniscus flow process shown in FIG. 1A, a higher volume fraction of gas/air is used in the interior compartment of the fluidic device (e.g., tube), which minimizes the ability to control the speed at which the fluid interface 32 can propagate across a surface to deposit the ECM polymer (e.g., to organize fibronectin). By way of example, the interior compartment 22 of fluidic device 20 has a total volume and a volume fraction of the liquid 24 to the total volume is less than or equal to about 0.55, for example, the volume fraction may be less than or equal to about 0.50, optionally, less than or equal to about 0.45, optionally less than or equal to about 0.4. In one example, where a total volume inside a tube is about 2.5 mL, an amount of liquid solution may be about 0.9 mL, so a volume fraction may be about 0.36, which will promote formation of a meniscus. In the meniscus flow process shown in FIG. 1A, flow is discontinuous and there is open fluid at interface 32. The ECM protein, like fibronectin, unfolds due to the surface tension force/shear stress that occurs. Notably, the meniscus 32 flows on and over most or all the surfaces in the interior region 22.

In comparison to the meniscus method, another method according to certain aspects of the present disclosure can produce a protein construct on or over a target region by a bubble flow technique in FIG. 1B. In certain aspects, the method produces a protein construct that may comprise a plurality of extracellular matrix protein fibrils, such as a fibrous fibronectin matrix, that is suspended across two surfaces with specificity of region, scale, and fibrillar alignment. The precipitation of the protein, such as a fibronectin matrix, can be manipulated by controlling the speed and position at which the fluid interface propagates in the process vessel to influence the contact interface and subsequent fibril formation.

Alignment of the extracellular matrix protein (e.g., fibronectin matrix) may be controlled by varying the volume of bubble within the vessel which can impact both the fluidics of the contact interface of the fluid propagating across the surface as well as the speed. The speed is believed to the be the predominant factor in governing fibril alignment. At a higher speed, unfolding fibronectin is restricted to the direction of the fluid movement. Owing to the linear conformation of fibronectin when unfolded and the locations of fibronectin binding domains, the natural tendency is a linear stacking of individual fibronectin fibrils (unfolded fibronectin). However, at a lower speed this allows for greater freedom for molecular motion and fluid turbulence. Hence, a deposited fibronectin matrix is formed with isotropic/misaligned topography.

As shown in FIG. 1B, a second fluidic device 50 may be a tube or other container that includes an interior region 52 that may be sealed and fluid-tight and contains both a liquid 54 and gas (e.g., air) 56. The gas 56 defines a bubble 60 at a two-phase interface 62 between the liquid 54 and gas 56. In the bubble flow process shown in FIG. 1B, a lower volume fraction of gas/air and greater volume fraction of liquid is used in the interior compartment of the fluidic device (e.g., tube) 50 to promote formation of a bubble 60. By way of example, the interior compartment 52 of fluidic device 50 has a total volume and a volume fraction of the liquid 54 to the total volume is greater than or equal to about 0.55, for example, the volume fraction may be greater than or equal to about 0.60, optionally greater than or equal to about 0.65, optionally greater than or equal to about 0.7. In one example, where a total volume inside a tube is about 2.5 mL, an amount of liquid solution may be about 1.6 mL, so a volume fraction may be about 0.64 that will promote formation of a bubble.

Two posts or supports 64 are disposed in the interior region 52 that support a porous polymer structure 66 or three-dimensional scaffold structure. By way of non-limiting example, as shown, a distance between the two supports 64 is about 6 mm. The arrow shows a direction that the bubble 60 advances through the interior region 22 of the fluidic device 20. As the bubble 60 advances past the first of the supports 64, it deposits a protein construct 70 that comprises a fibrillar matrix comprising the plurality of extracellular matrix protein fibrils. By shearing the two-phase interface 62 over the target surfaces (e.g., supports 64), fibrillar networks, such as insoluble fibronectin fibrils, are drawn out from the liquid comprising the protein (e.g., from a network of fibronectin that forms at the fluid interface 62). However, as shown, the protein construct 70 formed by the plurality of extracellular matrix protein fibrils is formed at the edge of the bubble 60 and thus where the bubble 60/interface 62 traverses from the first of the supports 64 to the second, the protein construct 70 will form a bridge from support to support 64 without depositing on the porous polymer structure 66. Notably, this bridge will extend between the respective supports 64 (e.g., 6 mm or more) and may be later removed from the supports 64 to form a free-standing structure/film. If coating of the porous polymer structure 66 is desired, the flow of bubble 60 may be directed to contact the porous polymer structure 66 where the protein construct 70 will then be deposited as desired. In the bubble flow process shown in FIG. 1B, flow is continuous and there is a closed fluid at interface 62. The ECM protein, like fibronectin, at least partially unfolds due to the surface tension force/shear stress that occurs. Notably, the surface of the bubble 60 contacts only selective surfaces within the interior region 22.

By changing the volume of the liquid comprising the extracellular matrix protein (e.g., fibronectin) solution in the tube, for example, increasing the solution volume to lower the volume fraction of gas/air in the interior compartment volume (e.g., tube), the fluid interface 62 becomes more like a bubble 60 entrapped in a solution, moving more freely as the fluidic device 50 (e.g., tube) is rotated and the bubble 60 flows against gravity by buoyancy. The fluid interface 62 of a bubble 60 allows ECM protein (e.g., fibronectin) matrix to be continuously organized, which can bridge across at least a near-centimeter-length (6 mm) gap between two support 64 surfaces. It is believed that the distance between supports can be much greater, for example, greater than or equal to about 5 cm, optionally greater than or equal to about 10 cm, and in other variations, optionally greater than or equal to about 15 cm. The fluid interface 62 of the bubble 60 thus serves as an organization front, or a guiding surface, for example, in the case of fibronectin due to its hydrophobic attraction towards air. Since air is hydrophobic, fibronectin aggregates at this interface with unfolding of fibronectin that exposes its hydrophobic domains. This facilitates the organization of fibronectin molecules with the fluid interface as a guide. As noted above, due to the higher volume fraction of gas/air, a bubble is not formed, but rather a meniscus. Here, a meniscus is a discontinuous/open liquid surface, unlike a bubble which is understood to be a continuous/closed liquid surface. The movement of meniscus can be visualized similar to a technique called molecular combing, which utilizes passing of air-liquid interface across the molecules of interest adsorbed onto a surface to impart mechanical forces derived from the surface tension of the interface. Because the meniscus is a discontinuous/open liquid surface, the molecule is statically bound to the surface that the meniscus propagates across. This is a distinction between the meniscus method and the bubble method according to certain aspects of the present disclosure. Additionally, as will be described further herein, by designing the geometry of fluidic device vessel itself, the contact points of the substrate and fluid can be manipulated to allow for regioselective deposition of the ECM (e.g., fibronectin) matrices as well as providing control of the topography of the resultant fibrils.

Thus, some of the advantages of utilizing a bubble-flow in a defined vessel, are the ability to: (1) induce continuous organization of an ECM protein (e.g., fibronectin) without needing additional support structure to further facilitate the organization steps (nucleation/unfolding/association/propagation), (2) manipulate the anisotropy/geometry of the fibrillar matrix in the protein construct, (3) control the contact point of the fluid interface to deposit ECM protein (e.g., fibronectin) matrix onto selective surfaces (4) facilitate scalability (arrays and large areas), (5) increase process throughput, and in certain aspects, and (6) deposit the ECM polymer (e.g., fibronectin) across gaps between support structures so that the protein construct can form a suspended or be removed from the support structures to form a “free-standing film.”

By way of comparison, where the extracellular matrix protein comprises fibronectin, the meniscus flow process employs an open/discontinuous air-liquid interface with hydrodynamic shearing of fibronectin. This results in a fluidic process that employs molecular combing for fibronectin, where movement of receding meniscus with respect to fibronectin and polymer scaffold helps facilitate the deposition of fibronectin. Adsorption of fibronectin onto the target surface occurs via initiation/nucleation. Unfolding of fibronectin occurs due to surface tension/shear force from the moving fluid interface. This results in organization of unfolding fibronectin and then is followed by continued organization at the surface. Finally, adsorption of organized fibronectin matrix onto the surface (termination) occurs.

Where the extracellular matrix protein comprises fibronectin, the bubble flow process employs a closed/continuous air-liquid interface for fibronectin organization. The mechanism involves adsorption of fibronectin onto the surface (initiation/nucleation), followed by unfolding of fibronectin due to surface tension/shear force from the moving fluid interface. Organization of unfolding fibronectin follows, where there is hydrophobic attraction of organizing fibronectin to the moving air-fluid interface. The continued organization of fibronectin occurs at the air-liquid interface with bubble flow and not on the surface as occurs with the meniscus flow. This enables organization and deposition of the fibronectin without a surface or substrate being disposed below it. The adsorption of organized fibronectin matrix occurs until termination of the process.

In certain aspects, the bubble process methods of the present disclosure permit the area occupied or coated by the deposited protein construct to have a large surface area, for example, a surface area of greater than or equal to about 3.3 cm², optionally greater than or equal to about 4 cm², optionally greater than or equal to about 5 cm², optionally greater than or equal to about 6 cm², optionally greater than or equal to about 7 cm², optionally greater than or equal to about 8 cm², optionally greater than or equal to about 9 cm², optionally greater than or equal to about 9.5 cm², and in certain variations, greater than or equal to about 9.6 cm². In other aspects, the bubble process methods of the present disclosure permit the deposited protein construct to have a large dimension, for example, greater than or equal to about 6 mm, optionally greater than or equal to about 10 mm, optionally greater than or equal to about 15 mm, optionally greater than or equal to about 20 mm, optionally greater than or equal to about 25 mm, optionally greater than or equal to about 30 mm, and in certain aspects, a dimension of greater than or equal to about 35 mm. In certain aspects, the protein constructs may be used in a large-scale engineered ECM (EECM) cell expansion platform based on a 3D fibrillar fibronectin network spanning that spans over centimeters, where the extended fibrillar networks are formed by shearing dilute fibronectin solutions over tessellated polymeric scaffolds, which are conveniently prepared by commercial 3D printers.

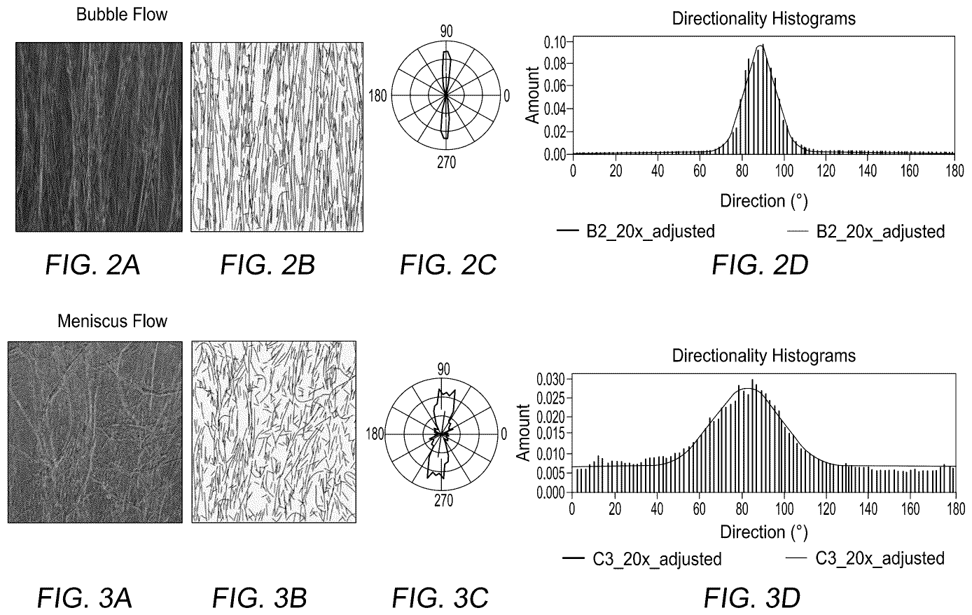

Protein constructs of an ECM protein (e.g., fibronectin) comprising a plurality of extracellular matrix protein fibrils formed by a bubble flow method in FIGS. 2A-2D versus via a meniscus flow method in FIGS. 3A-3D are compared. In FIG. 2A, a scanning electron microscope shows a highly aligned and anisotropic fibronectin matrix formed via a bubble method like that shown in FIG. 1B with a representative schematic drawing in FIG. 2B showing the alignment of fibers. By using ImageJ analysis, an average direction or alignment of the major longitudinal direction of the fibronectin fibrils deposited by the bubble method is shown in FIG. 2C. FIG. 2D shows directionality of histograms for the deposited fibronectin matrix fibrils via the bubble flow method, for example, providing a direction and a dispersion, where direction is shown as a center of the gaussian curve (with goodness of fit of the gaussian curve) and dispersion is a standard deviation of the directionality. An amount may be a sum of the histogram from center−standard to center+standard divided by a total sum of the histogram. Meanwhile, FIG. 3A shows a scanning electron microscope of the fibronectin matrix formed via a meniscus method like that shown in FIG. 1A with a representative schematic drawing in FIG. 3B showing the lack of alignment/misalignment of deposited fibers. An average direction or alignment of the major longitudinal direction of the fibronectin fibrils deposited by the meniscus method is shown in FIG. 3C, while FIG. 3D shows directionality of histograms for the deposited fibronectin matrix fibrils via the meniscus method. When comparing FIGS. 2C and 3C and then FIGS. 2D and 3D, the anisotropy and alignment of the anisotropy of the longitudinal axes of the deposited fibronectin fibers via bubble method as compared to the meniscus method are apparent. By “anisotropic” it is meant each of the fibers or fibrous structures formed has an evident longitudinal axis and the respective longitudinal axes of the plurality of fibers/fibrils are substantially aligned. If not otherwise understood, by substantially aligned, it is meant that greater than 90% of the longitudinal axes of fibers/fibrils have a common or aligned direction that is within about ±25°, optionally about ±20°, and in certain variations, optionally about ±15°of a central direction (for example, a central direction along a 90° to 270° orientation, as shown in FIG. 2C).

According to certain aspects of the present disclosure, a method of making a cellular support system is contemplated. The method may comprise passing a bubble comprising gas through a liquid comprising an extracellular matrix protein contained in a fluidic device. The gas may be air in certain variations. The extracellular matrix protein is initially dissolved as a solubilized protein in the liquid. The bubble is passed over a target within the fluidic device. The gas/liquid interface causes the extracellular matrix protein to organize and form a deposited protein construct comprising a plurality of extracellular matrix protein fibrils on or near the target. In certain aspects of the present disclosure, the fluidic device in which the method is conducted may have a total volume (e.g., interior volume) and a volume fraction or ratio of the liquid to the total volume is greater than or equal to about 0.55, or any of the other ratios specified above, to facilitate formation of a bubble. After conducting the methods described herein, the dissolved extracellular matrix protein is transformed into an insoluble protein construct, notably without the need for use of cells to do so. The deposited protein construct may be a suspended bridge, a suspended or free-standing film, and/or in alternative aspects, a coating, but serves to of support cells and promote three-dimensional cellular growth. By “promoting” cell growth, cell proliferation, cell differentiation, cell repair, or cell regeneration, it is meant that a detectable increase occurs in either a rate or a measurable outcome of such processes when the cellular support system is present as compared to a cell or organism's process in the absence of the cellular support system, for example, conducting such processes naturally. By way of example, as appreciated by those of skill in the art promoting cell growth in the cellular support system may increase a growth rate of target cells or increase a total cell count of the target cells, when compared to cell growth or cell count of the target cells in the absence of such a cellular support system.

By “supporting” cell growth, cell proliferation, cell differentiation, cell repair, or cell regeneration, it is meant that the cellular support system provides a physical substrate for one or more target cells that enhances target cell growth, vitality, proliferation, differentiation, repair, or regeneration, by way of non-limiting example. As appreciated by those of skill in the art, the cellular support system may both support and promote the growth, vitality, proliferation, differentiation, repair, and/or regeneration processes of one or more target cells in vitro, ex vivo, or in vivo, for example. The cellular support system thus can serve a role as a cellular scaffold structure that supports and/or promotes target cell growth, target cell proliferation, target cell differentiation, target cell repair, and/or target cell regeneration in three-dimensions, in contrast to the support and growth on conventional two-dimensional planar or two-dimensional scaffold surfaces. The cellular support systems provided by certain aspects of the present disclosure can be employed to promote growth of one or more target cells in a predetermined three-dimensional pattern.

The final product may comprise an insoluble ECM protein molecule formed into a complex folded shape. The complexly folded molecule is insoluble in water and aqueous solutions, preferably is fibrillar, and is also biologically active to cells in the environment. Here, because the proteins present in the deposited protein construct are insoluble, the cryptic binding sites can be revealed despite being fully-defined and cell-free. In certain aspects, the process, such as bubble flow method of deposition can provide control over a speed of the ECM protein (e.g., fibronectin) deposition, which provides for differential opening of epitopes within the protein that may facilitate different interactions with the cells as they grow on the deposited protein product/construct. The deposited protein construct can be lyophilized for long-term storage, transport, and analysis. The deposited protein construct can also last in an aqueous solution without degradation, as fabricated, for over one week. In certain variations, the plurality of extracellular matrix protein fibrils in the deposited construct has an anisotropic orientation. In certain alternative variations, the deposited protein construct may further comprise additional materials, such as at least one glycan, which may comprise a glycosaminoglycan like hyaluronic acid, for example, like the materials described in PCT Patent Application No. PCT/US2023/021147 filed on May 5, 2023, entitled “Engineered Fibrillar Extracellular Matrix Networks for Three-Dimensional (3D) Cellular Support Systems,” the relevant portions of which are incorporated herein by reference.

In certain variations, the methods may be conducted where the target comprises a three-dimensional scaffold structure comprising at least one void and the deposited protein construct spans over the at least one void. Where the protein construct is deposited on a three-dimensional scaffold structure, it may comprise a polymer and define the three-dimensional cellular support system. Where used, the three-dimensional scaffold structure comprises at least one void and typically a plurality of voids. A void as used herein is an open volume formed within a solid or semi-solid material. Voids may include pores, surface features, holes, openings, roughness, or topography, by way of example. For example, while a void may be a pore in a porous material, a void may also include spaces defined between adjacent features of a rough surface. A rough surface that defines a void may have an average surface roughness (Ra) value of greater than or equal to about 0.025 micrometers. The shape of the voids or pores is not limited but may be any number of shapes including those having a cross-sectional shape of a square, a circle, a rectangle, an oval, a parallelogram, a triangle, or any other regular or irregular cross-sectional or three-dimensional shape. In certain aspects, the polymeric scaffold may be a tessellated polymeric scaffolds, which may be made by additive manufacturing/3D printing.

The overall shape of the three-dimensional scaffold structure may be of any shape, including customized shapes and sizes, and is not correlated with the shape of the void(s). By way of non-limiting example, a square-shaped three-dimensional scaffold structure can include a plurality of circular voids/pores. In certain aspects, the three-dimensional scaffold structure may be an implantable device that is used in vivo and thus introduced into a subject, such as a human. In other aspects, the three-dimensional scaffold structure may be used as for cellular or tissue growth ex vivo or in vitro.

In certain variations, the three-dimensional scaffold structure comprises a plurality of voids. Such voids may be of the same shape and/or in a repeating pattern, but need not be of the same shape or size. Thus, a shape of each void need not be the same and there is no limitation on the number of voids/pores having the same shape. For example, a scaffold structure can have four voids, one having a triangular cross-sectional shape, two or more having a circular cross-sectional shape, and one having a rectangular or square cross-sectional shape.

In certain aspects, the three-dimensional scaffold structure is a porous material having a plurality of pores. The plurality of pores may optionally be open pores that are interconnected with one another. In certain variations, a pore density may be greater than or equal to about 1 void or pore regions/scaffold material to less than or equal to about 1.0×10¹² void or pore regions/scaffold material. As will be appreciated by those of skill in the art, as the scaffold structure becomes bigger, more pores are added, continuously making the pores smaller and also adding more pores. In certain aspects, the percent open area of the scaffold structure is maximized, so that the scaffold structure may have greater than or equal to about 0.0001% by volume of voids, optionally greater than or equal to about 0.001% by volume of voids, optionally greater than or equal to about 0.01% by volume of voids, optionally greater than or equal to about 0.1% by volume of voids, optionally greater than or equal to about 1% by volume of voids, optionally greater than or equal to about 10% by volume of voids, optionally greater than or equal to about 20% by volume of voids, optionally greater than or equal to about 30% by volume of voids, optionally greater than or equal to about 40% by volume of voids, optionally greater than or equal to about 50% by volume of voids, optionally greater than or equal to about 60% by volume of voids, optionally greater than or equal to about 70% by volume of voids, optionally greater than or equal to about 80% by volume of voids, optionally greater than or equal to about 90% by volume of voids, optionally greater than or equal to about 91% by volume of voids, optionally greater than or equal to about 92% by volume of voids, optionally greater than or equal to about 93% by volume of voids, optionally greater than or equal to about 94% by volume of voids, optionally greater than or equal to about 95% by volume of voids, and in certain preferred aspects, optionally greater than or equal to about 96% by volume of voids.

When present in the three-dimensional scaffold structure, the void(s) may have a major dimension of greater than or equal to about 0.025 micrometers, optionally greater than or equal to about 0.1 micrometers to less than or equal to about 5 centimeters, optionally greater than or equal to about 0.5 micrometers to less than or equal to about 4 centimeters, optionally greater than or equal to about 1 micrometers to less than or equal to about 3 centimeters, and in certain variations, optionally greater than or equal to about 5 micrometers to less than or equal to about 2 centimeters. By major dimension, it is meant the greatest dimension of the void and may be, for example, length, width, or diameter. In certain variations, a void in the form of a pore may have a length of greater than or equal to about 0.1 mm (100 μm) to 1 millimeter for cell culture applications.

A simplified non-limiting three-dimensional scaffold structure as part of a three-dimensional cellular support system is shown in co-owned U.S. Pat. No. 11,479,753 to Ramacharan et al., the relevant portions of which are incorporated by reference. An overall length and width of the three-dimensional scaffold structure is unrestricted and will be dictated by the intended application. By way of example only, scaffold structure may have dimensions of 10 millimeters by 8 mm with a thickness of 0.6 millimeters and have a void/pore that is a single square opening with a side length of 5 millimeters, which may be advantageous for use in laboratory three-dimensional cell culture since this size fits into a centrifuge tube and the well of a 24-well plate. Suitable scaffold structures may have a plurality of interior voids that have a representative rectangular shape in a regular repeating three-dimensional mesh pattern; however, as noted above the voids are not limited to these shapes or positions and may have different shapes and arrangements within the scaffold structure. The scaffold structure includes walls that define and surround each void. Interior surfaces of the walls thus define voids. Further, any of the interior surfaces of the voids may have a coating, such as a protein-containing coating, disposed thereon. Each void has at least one suspended protein construct, in the form of a suspended bridge, spanning from a first interior surface to a second interior surface across the void and thus anchored at each end on each respective interior surface. While not every void may have a suspended protein bridge, in certain variations, each void may have multiple suspended protein bridges, including two, three, or multiple bridges. For example, each void may have one bridge to thousands of bridges per void, for example, in certain variations, from greater than or equal to about 10 bridges to less than or equal to about 1,000 bridges. In certain aspects, the suspended protein bridges 70 may be a plurality of suspended bridge structures numerous enough to form a suspended protein mesh across the void. It should be noted that while the orientation and position of the protein bridges 70 is shown generally to be the same, they may have different orientations, different anchor points, and therefore different lengths within each void. Further, different voids within the scaffold may have different numbers or densities of suspended protein bridge structures.

In certain aspects, a wall defined between the plurality of voids has an average thickness of greater than or equal to about 0.025 micrometers. In certain variations, a wall defined between the plurality of voids has an average thickness of greater than or equal to about 0.5 micrometers, optionally greater than or equal to about 1 micrometer, and in certain aspects, optionally greater than or equal to about 5 micrometers. In certain variations, a wall may have an average thickness of greater than or equal to about 0.5 micrometers to less than or equal to about 300 micrometers, optionally greater than or equal to about 1 micrometers to less than or equal to about 300 micrometers, optionally greater than or equal to about 5 micrometers to less than or equal to about 300 micrometers, optionally greater than or equal to about 10 micrometers to less than or equal to about 300 micrometers, and in certain variations, optionally greater than or equal to about 100 micrometers to less than or equal to about 300 micrometers. In certain aspects, a depth or major dimensions of each void/pore 60 is less than or equal to a thickness of a wall of the scaffold structure.

In various aspects, the three-dimensional scaffold structure is formed of a biocompatible material. By “biocompatible,” it is meant that a material or combination of materials can be contacted with cells, tissue in vitro or in vivo, or used with mammals or other organisms and has acceptable toxicological properties for contact and/or beneficial use with such cells, tissue, and/or animals. For instance, in certain aspects, a biocompatible material may be one that is suitable for implantation into a subject without adverse consequences, for example, without substantial toxicity or acute or chronic inflammatory response and/or acute rejection of the material by the immune system, for instance, via a T-cell response. It will be recognized, of course, that “biocompatibility” is a relative term, and some degree of inflammatory and/or immune response is to be expected even for materials that are highly compatible with living tissue. However, non-biocompatible materials are typically those materials that are highly toxic, inflammatory and/or are acutely rejected by the immune system, e.g., a non-biocompatible material implanted into a subject may provoke an immune response in the subject that is severe enough such that the rejection of the material by the immune system cannot be adequately controlled, in some cases even with the use of immunosuppressant drugs, and often can be of a degree such that the material must be removed from the subject. In certain aspects, biocompatible materials are those that are approved for use in humans by an appropriate regulatory agency, such as the Federal Drug Administration (FDA) in the United States; the European Commission (EC)/European Medicines Agency (EMEA) in Europe; or Health Products and Food Branch (HPFB) in Canada.

In certain variations, the scaffold structure is formed of a biodegradable material, while in other variations; the scaffold structure is formed of a non-biodegradable material. A biodegradable material may dissolve or disintegrate ex vivo or in vivo. “Dissolving” refers to physical disintegration, erosion, disruption and/or dissolution of a material and may include the resorption of a material by a living organism. Dissolution or erosion occurs when the material is exposed to a solvent comprising a high concentration of water, such as growth or culture media, serum, blood, saliva, bodily fluids, and the like. In certain aspects, the three-dimensional scaffold structure optionally comprises a combination of biocompatible materials, like a combination of polymer materials.

The material forming the scaffold structure may include a biofunctional active ingredient that is released as the biodegradable material dissolves or disintegrates. Biofunctional active ingredients or agents may include pharmaceutical active ingredients, proteins, peptides, growth factors, biofactors, imaging agents by way of non-limiting example. The biofunctional active ingredient may be dispersed within the material that forms the scaffold. Inclusion of a biofunctional active ingredient or agent may be preferred where the scaffold material is biodegradable thus allowing the release of various compounds of interest such as pharmaceuticals or imaging agents.

The three-dimensional scaffold structure can be made of a wide variety of materials, including inorganic and organic biocompatible materials. The three-dimensional scaffold structure may be formed from a material selected from the group consisting of: a metal material, a ceramic material, a glass material, a polymeric material, a composite material (having a polymeric material and a reinforcement material), a ceramic material, a biologically-derived material (a material derived from a biological source, such as cellulose or paper), and combinations thereof and need not be uniform or homogeneous throughout the scaffold structure (e.g., there may be distinct regions of the scaffold with different compositions). Certain polymeric and composite materials may be biodegradable, while other polymeric, composite, and metal materials are not biodegradable. Specifically, biocompatible polymer materials, such as biodegradable or non-biodegradable polymers, synthetic or natural polymers can be used.

By way of example, suitable polymers include polyethers, such as a polyethylene oxide (PEO), polyoxyethylene glycol or polyethylene glycol (PEG), biodegradable polymers such polyesters like polylactic acid, polycaprolactone, polyglycolic acid, poly(lactide-co-glycolide polymer (PLGA), poly(lactide-co-caprolactone), and copolymers, derivatives, and combinations thereof. Suitable water-soluble and/or hydrophilic polymers, which are biocompatible, include cellulose ether polymers like hydroxypropyl methyl cellulose (HPMC), hydroxypropyl cellulose (HPC), hydroxyethyl cellulose (HEC), methyl cellulose (MC), carboxymethyl cellulose (CMC), and combinations thereof. Various polysaccharides include starches such as maltodextrin, amylose, corn starch, potato starch, rice starch, tapioca starch, pea starch, sweet potato starch, barley starch, wheat starch, modified starch (e.g., hydroxypropylated high amylose starch), and the like.

In certain variations, the three-dimensional scaffold structure comprises a polymer and is formed from a polymeric precursor or is a polymer selected from the group consisting of: polylactic acid, polyglycolide, polycaprolactone, poly(lactide-co-glycolide), poly(lactide-co-caprolactone), polyethylene glycol, polydimethylsiloxane, polyurethanes, polyolefins, polyamides, celluloses, lignins, starches, biodegradable polyesters, polystyrene, and combinations thereof.

Other water-soluble polymers among those useful herein include, without limitation, sodium alginate, carrageenan, xanthan gum, gum acacia, Arabic gum, guar gum, pullulan, agar, chitin, chitosan, pectin, karaya gum, locust bean gum, various polysaccharides; starches such as maltodextrin, amylose, corn starch, potato starch, rice starch, tapioca starch, pea starch, sweet potato starch, barley starch, wheat starch, modified starch (e.g., hydroxypropylated high amylose starch), dextrin, levan, elsinan and gluten; and proteins such as collagen, whey protein isolate, casein, milk protein, soy protein, keratin, and gelatin.

The choice of the scaffold structure material can vary based upon the intended application of the cell culture system. When used to facilitate three-dimensional culture of adherent cells, polymeric materials may be advantageous by providing a softer mechanical environment. These include, without limitation, polylactic acid, polyglycolide, polyethylene glycol, polycaprolactone, starches, biodegradable polyesters, co-polymers such as poly(lactide-co-glycolide) and poly(lactide-co-caprolactone), and non-degradable materials such as polystyrene. If intended for long-term use, or as an implant with intention of surgical removal, a metal scaffold may be preferred for rigidity, stability, and lack of degradability.

In certain aspects, the three-dimensional scaffold structure can be chemically anisotropic. This may be achieved, for example, by reacting or tethering different functional groups to localized areas of select surfaces of the scaffold structure. Chemical anisotropy can also be realized by compartmentalizing the bulk material of the scaffold structure into domains of varying chemical composition.

In certain aspects, the three-dimensional scaffold structure may be custom-made or commercially available. Scaffolds can be custom made with desired opening sizes and shape. For polymeric scaffolds, fabrication options include without limitation, solid free form fabrication, additive manufacturing (e.g., 3D printing, 3D jet writing, direct writing), and extrusion. Metal scaffolds, again without limitation, can be milled, machined, photochemically etched, or formed via additive manufacturing (e.g., direct metal laser sintering).

A protein loading density in the cellular support system may greater than less than or equal to about 8 milligrams of protein per milligram of the polymer in the cellular support system. By way of example, a suitable protein loading density may be 100 micrograms of protein per milligram of polymer.

Global stiffness of the protein-scaffold structure and compressibility of the suspended protein bridges can be tuned by modifying the scaffold composition, and/or modulating pore shape and size. Larger voids/pores in the scaffold structure will favor greater compressibility, whereas smaller voids/pores will favor stiffer protein bridges.

Larger pores given a fixed scaffold length and width also reduces interactions between the protein and solid scaffold surfaces, tending to maintain the protein in its native conformation. This also minimizes exposure of adherent cells (if any) to synthetic scaffold material (if synthetic) to preserve cell phenotype and maintain natural cell behavior.

The suspended protein construct can provide cells with an ECM-like network of protein which is susceptible to remodeling by the cells, allowing for study of cell migration and metastasis. When cells secrete insoluble proteins to form their microenvironment, they are also revealing biologically active cryptic binding sites on the protein that are otherwise inaccessible to cells when the protein is solubilized. For example, cellular fibronectin (cFN) is found in many isoforms, each exposing the mechano-sensitive FNIII domain, which is key to fibrillogenesis and matrix assembly. cFN is assembled by cells that bind to the protein through integrin-based structures. Translocation of these integrins on the cell surface imparts tension that unfolds FNIII domains, exposing self-association binding sites that allow interactions between fibronectin molecules leading to cFN fibrillogenesis. Here, because the proteins present in the protein construct/bridge are insoluble, the cryptic binding sites can be revealed despite being fully-defined and cell-free.

In certain variations, the methods may be conducted where the target comprises a three-dimensional scaffold structure formed from a material selected from the group consisting of: a metal material, a polymeric material, a composite material, a ceramic material, a biologically-derived material, and combinations thereof. In certain variations, the three-dimensional scaffold structure comprises a polymer and is formed from a polymeric precursor or is a polymer selected from the group consisting of: polylactic acid, polyglycolide, polycaprolactone, poly(lactide-co-glycolide), poly(lactide-co-caprolactone), polyethylene glycol, starches, polydimethylsiloxane, polyurethanes, polyolefins, polyamides, celluloses, lignins, biodegradable polyesters, polystyrene, and combinations thereof.

In certain other variations, the methods may be conducted where the target comprises at least one surface in the fluidic device such that the deposited protein construct coats the at least one surface. In certain variations, the target comprises a first support and a second support, so that the passing of the bubble occurs from the first support to the second support and the deposited protein construct bridge or film spans from the first support to the second support. The methods may further comprise removing the protein construct from the first support and the second support for form a free-standing film.

In certain other variations, the methods may include passing the bubble inside the fluidic device by moving the fluidic device. The moving of the fluidic device may be selected from the group consisting of: tumbling/rotation, tipping, orbital rotation, spinning, vibration, shaking, and combinations thereof. Generally, mechanical stimuli from these or potentially other processes can be used to directly or indirectly induce at least partial unfolding of fibronectin. In certain aspects, the passing of the bubble may occur for greater than or equal to about 5 minutes to less than or equal to about 96 hours.

Temperature during incubation must be selected carefully to prevent protein denaturation that can occur when temperatures are too high. Again, different proteins and substrates may result in different optimal incubation temperatures. In one aspect, a suitable range of incubation temperatures is greater than or equal to about 4 °C to less than or equal to about 40 °C. In certain aspects, it may be desirable to incubate in sequences of different temperatures for the duration of the contact time.

Some proteins require a higher concentration in solution. The exact concentration will vary based on the solution surface tension, ionic strength, and desired protein loading, but in certain aspects, the liquid comprising extracellular matrix protein is a protein solution having a concentration of protein from greater than or equal to about 0.01 mg/mL to less than or equal to about 2.5 mg/mL, optionally from greater than or equal to about 0.01 mg/mL to less than or equal to about 1 mg/mL.

In summary, the bubble methods contemplated by the present disclosure decouple fibrillar extracellular matrix protein (e.g., fibronectin matrix) from a support structure allowing for various surfaces to be coated and enabling numerous technological advancements. The utilization of this new bubble flowing technique with custom vessel geometries allows for regioselectivity, enabling applications such as the coating of arrays, complex device fabrication, large areas, as well as high throughput arrays. The new methods can provide various advantages for biological applications, in which the following are important factors: substrate mechanics, fibril alignment/geometry, continuous cell seeding/recovery, dynamic cell culture (bioreactor), large scale culture, and/or micro/high-throughput arrays. These culture substrate characteristics are important in a wide range of applications in regenerative tissue engineering, stem cell engineering, wound healing, tumor cell culture, and the like. By way of non-limiting example, the assembled protein matrices have been used is in cardiac engineering, tumor microenvironment engineering, and neural engineering. Further, high throughput arrays allow for combinations of extracellular protein and supporting cell types to be explored in order to recapitulate healthy/pathological states of cardiac, neural, skin and/or tumor microtissues. These arrays can also be used to screen drugs for toxicity (healthy) or efficacy (pathogenic) to better predict outcome using this humanized in vitro approach, which is highly attractive for pharmaceutical applications.

Furthermore, dynamic cell cultures, such as minibioreactors/actuating systems can facilitate maturation of tissue constructs, which is especially important in applications like cardiac engineering or for obtaining sufficient cells for developing cancer cell-based vaccines, which are otherwise challenging and may require additional cytokine and checkpoint inhibitors for optimal therapeutic activity. Optimized small-scale/microtissues could then be scaled using larger reactor systems or traditional static culture (well plates/petri dishes) given the highly modular nature of this system. That is of particular interest in tumor modeling, where the ex vivo growth/expansion of primary patient cells is very challenging but crucial in developing rapid, efficacious personalized medicines (for example, in immuno-oncology). For example, this may be utilized in chimeric antigen receptor (CAR)-T therapy for treating certain hematologic cancers and solid cancers, as well as “armed” autologous whole-tumor cell immunotherapies. Thanks to the benefits of dynamic cell cultures, these therapies present neoantigen targets for therapeutic immune effector cells and enhance antitumor immune response through molecular modulation. Additionally, optimized, large-scale cardiac, neural or skin constructs could be employed as a tissue scaffold patch to facilitate the regeneration of normal tissue/function.

In certain aspects, the present disclosure contemplates a cellular support that comprises a deposited protein construct comprising a plurality of extracellular matrix protein fibrils aligned in an anisotropic orientation, meaning the fibrils or fibers are highly aligned with one another, as defined above. The cellular support comprising the deposited protein construct is capable of supporting cells and promotes three-dimensional cellular growth. In certain variations, the extracellular matrix protein is selected from the group consisting of: fibronectins, laminins, collagens, tenascins, elastin, vitronectin, periostin, and combinations thereof, and in certain particular variations, the extracellular matrix protein comprises fibronectin.

The cellular support may further comprise a three-dimensional scaffold structure comprising at least one void, where the deposited protein construct spans over the at least one void. The three-dimensional scaffold structure is formed from a material selected from the group consisting of: a metal material, a polymeric material, a composite material, a ceramic material, a biologically derived material, and combinations thereof. The three-dimensional scaffold structure comprises a polymer and is formed from a polymeric precursor or is a polymer selected from the group consisting of: polylactic acid, polyglycolide, polycaprolactone, poly(lactide-co-glycolide), poly(lactide-co-caprolactone), polyethylene glycol, starches, polydimethylsiloxane, polyurethanes, polyolefins, polyamides, celluloses, lignins, biodegradable polyesters.

In certain further aspects, the present disclosure provides methods of expanding growth of cells by using such a cellular support. For example, the present disclosure contemplates a method of expanding three-dimensional growth of cells, which may comprise introducing cells to a cellular support comprising a deposited protein construct comprising a plurality of extracellular matrix protein fibrils aligned in an anisotropic orientation; and growing the cells on the deposited protein construct in three-dimensions. In certain aspects, the cells may be cancer cells. In one aspect, the method is used for an expansion of cancer cells for the construction of cancer immunotherapies. In other aspects, the cells may be cell mixtures obtained from human tumors. In a further aspect, the cells may be combinations of cells obtained from human tumors and support cells. In certain other aspects, the cells are stem cells. For example, in one variation, the cells are induced pluripotent stem cells. In yet other variations, the cells are adult cells. The adult cells may be selected from a group consisting of: cardiomyocytes, neurons, fibroblasts, and combinations thereof. In certain variations, the method is optionally used for construction of autologous whole-tumor cell immunotherapies. In other variations, the method may be used for construction of chimeric antibody receptor (CAR) T-cell therapies. In yet other variations, the method may be used for construction of organoid structures. In further variations, at least one cell may be genetically modified during or after expansion on the cellular support.

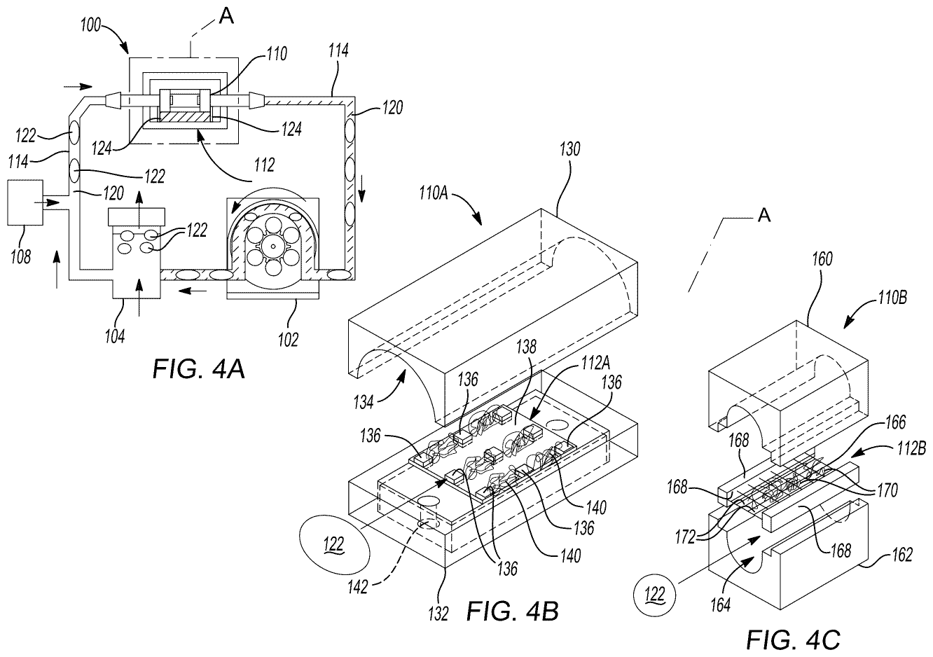

FIGS. 4A-4C show one non-limiting example of a system 100 in which a bubble flow method of making a cellular support system may be conducted. In certain aspects, such a system 100 may be used to facilitate continuous or semi-continuous bubble flow processing. The system 100 includes a pump (e.g., peristaltic pump) 102, a bubble chamber 104, and a reaction chamber 106 that holds a fluidic device 110 in which at least one target 112 is contained. A gas pump 108 injects a gas, such as air, that may be intermittently injected into a liquid 120 comprising an extracellular matrix protein solution to form a plurality of bubbles 122. The system 100 includes a fluid flow conduit 114 that connects the pump 102, bubble chamber 104, reaction chamber 106, and into which gas pump 108 injects gas to generate the plurality of bubbles 122. A direction of flow in the fluid flow conduit 114 is shown by the arrows in FIG. 4A. In the bubble chamber 104, a reservoir is formed that can store excess liquid 120 and bubbles 122. The liquid 120 and the bubbles 122 are then directed past the gas pump 108 and into the reaction chamber 106 where the fluidic device 110 is disposed. The fluidic device 110 is a removable component that may be readily replaced and provides versatility to the system 100. For example, the fluidic device 110 may be removably seated in one or more holder components 124 in the interior region of the reaction chamber 106. The continuous flow of bubbles 122 passing by can serve to form a deposited protein construct comprising a plurality of extracellular matrix protein fibrils on or near the target 112. As shown in FIGS. 4B and 4C, by way of non-limiting example, two distinct fluidic (e.g., microfluidic) devices, a first microfluidic device 110A and a second microfluidic device 110B are shown.

In certain variations, by microfluidic it is meant that the device has one or more interior regions that are on a microscale. The term “microscale” encompasses “nanoscale,” as well. In certain variations of the present teachings, a microfluidic feature has at least one spatial dimension that is less than about 1,000 micrometers (i.e., 1 mm), optionally less than or equal to about 500 μm (i.e., 0.5 mm), optionally less than or equal to about 100 μm (i.e., 0.1 mm), optionally less than or equal to about 50 μm (i.e., 0.05 mm), optionally less or equal to about 10 μm, optionally less or equal to about 5 μm, optionally less or equal to about 1 μm (i.e., 1,000 nm), optionally less than or equal to about 0.5 μm (i.e., 500 nm), and in certain aspects less than or equal to about 0.1 μm (i.e., 100 nm).

In FIG. 4B, the first microfluidic device 110A includes a lower component 130 and an upper component 132 that mate together to form a fluid-tight seal that defines an interior region 134. The lower component 130 and the upper component 132 may be formed of a polydimethylsiloxane (PDMS) material, by way of non-limiting example. The bubble 122 and liquid 124 flow through the interior region 134 of the first microfluidic device 110A (see arrow in FIG. 4B showing the direction of the bubble as it will flow through the interior region 134). As shown, a first target region 112A comprises a plurality of support posts 136 disposed on a substrate 138. In certain variations, the support posts 136 and the substrate 138 may be formed of a polydimethylsiloxane (PDMS). The support posts 136 can serve as initiation sites for creating a deposited protein construct 140 comprising a plurality of extracellular matrix protein fibrils as the bubble(s) 122 may over them. In this manner, the deposited protein construct 140 can span between respective support posts 136. This deposited protein construct 140 may be used as tissue culture platform, by way of non-limiting example. The first microfluidic device 110A may also have an opening or aperture 142 that can receive a vertical projection that may be used to seat the microfluidic device 110A in the holder component 124 and/or for mechanical actuation. Once formation of the deposited protein construct 140 is completed, the first target region 112A may be replaced with a new substrate 138 having support posts 136 for additional deposition of the extracellular matrix protein via the bubble flow method. Notably, only the substrate 138 with support posts 136 may be replaced or the entire first microfluidic device 110A may be replaced, depending on the application.

Another variation of the second microfluidic device 110B is shown in FIG. 4C which like the first microfluidic device 110A may be removably seated in one or more holder components 124 in the interior region of the microfluidic reaction chamber 106. The second microfluidic device 110B also includes a lower component 160 and an upper component 162 that mate together to form a fluid-tight seal that defines an interior region 164. Again, the lower component 160 and the upper component 162 may be formed of a polydimethylsiloxane (PDMS) material, by way of non-limiting example. The bubble 122 and liquid 124 flow through the interior region 164 of the second microfluidic device 110B (see arrow in FIG. 4B showing the direction of the bubble as it will flow through the interior region 164). As shown, a second target region 112B comprises a three-dimensional polymeric scaffold 166 seated within lateral supports 168. In certain variations, the polymeric scaffold 166 may be formed of polycaprolactone (PCL), while the lateral supports 168 may encapsulate the sides of the polymeric scaffold 166 so that it can be seated within the interior region 164 so that liquid 120 and bubbles 122 may pass through it. The surfaces of the polymeric scaffold 166 having one or more voids 172 can serve as initiation sites for creating a deposited protein construct 170 comprising a plurality of extracellular matrix protein fibrils. In this manner, the deposited protein construct 170 can span between surfaces of each void 172 in the polymeric scaffold 166. This deposited protein construct 170 may be used as tissue culture platform, by way of non-limiting example. Once formation of the deposited protein construct 170 is completed, the second target region 112B may be replaced with a new polymeric scaffold 166 with lateral supports 168 so that additional deposition of the extracellular matrix protein via the bubble flow method can occur. Notably, only the polymeric scaffold 166 with the lateral sides 168 may be replaced or the entire second microfluidic device 110B may be replaced, depending on the application.

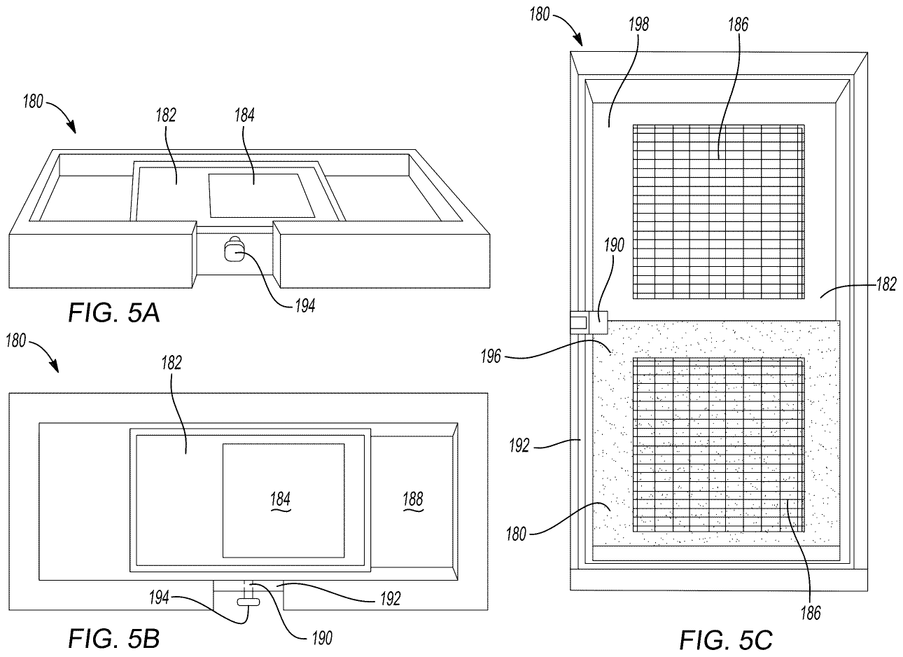

FIGS. 5A-5C show a fluidic (e.g., a microfluidic) device 180 prepared in accordance with certain aspects of the present disclosure. The device 180 includes a holding component 182 that may be a platform or substrate that has one or more open regions 184 that may receive one or more three-dimensional polymeric scaffolds 186. The polymeric scaffolds 186 may be any of those described previously above, for example, a polycaprolactone (PCL) porous scaffold. The open region(s) 184 of the holding component 182 may have dimensions that correspond to that of the three-dimensional polymeric scaffold 186 to be received therein, so that the scaffold 186 can be seated therein. The fluidic device 180 may have a recessed region 188 configured to receive liquid and a gas. The fluidic device 180 may have an upper lid (not shown) that will seal the recessed region 188 having the polymeric scaffold 186 disposed therein. One or more ports 190 may be formed in a wall of the 192 of the fluidic device 180 for injecting a liquid comprising a protein and if necessary, additional gas into the sealed recessed region 188. The various components of the fluidic device 180, aside from the scaffold 186, may be formed from a polymeric material, like polydimethylsiloxane (PDMS). In FIG. 5C, two polymeric scaffolds 186 are disposed in the holding component 182. In FIG. 5C, a liquid 196 comprising the protein (at the bottom half) and a gas 198 (at the top half) are charged in the recessed region 188.

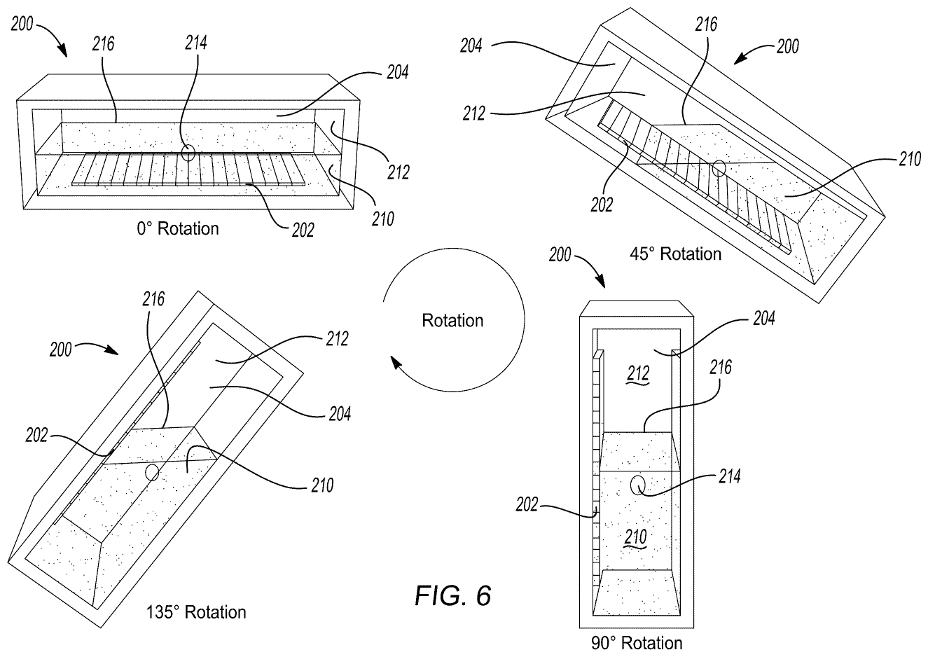

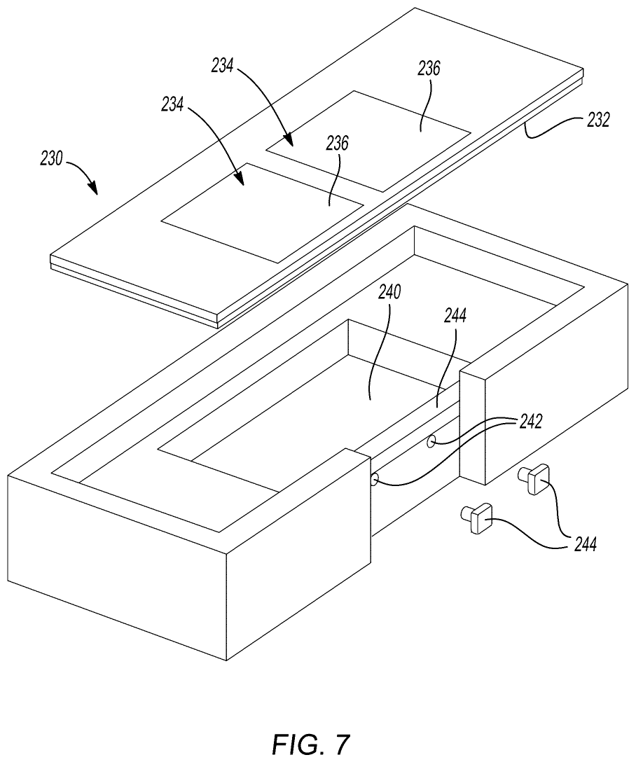

FIG. 6 shows a fluidic device 200 similar to that described in the context of FIGS. 5A-5C being rotated or tumbled to coat three-dimensional scaffolds in accordance with certain aspects of the present disclosure. Any fluidic device that can be used in a rotating or tumbling movement for forming the deposited extracellular matrix protein construct may be considered to have a “tumbling design.” Namely, the process shown in FIG. 6 provides a fluidic device that can be used with the meniscus flow process described above, for example, in the context of FIG. 1A. However, as will be appreciated by those of skill in the art, if the volume of liquid as compared to gas is increased, the fluidic device 200 may be used in a bubble flow process as described above in the context of FIG. 1B. The fluidic device 200 includes one or more three-dimensional polymeric scaffolds 202. An interior region 204 is configured to receive liquid comprising a liquid 210 comprising a protein and a gas 212. One or more ports 214 may be formed in a wall of the fluidic device 200 for injecting a liquid comprising a protein and if necessary, additional gas into the sealed interior region 204. The various components of the fluidic device 200, aside from the scaffold 202, may be formed from a polymeric material, like polydimethylsiloxane (PDMS). At 0° rotation, the liquid 210 is at a bottom half of the interior region 204, while the gas 212 is on a top half. The scaffold 202 is thus disposed in the liquid 210. The fluidic device 200 may be rotated to 45° where a lower portion of the scaffold 202 remains in the liquid 210 while an upper portion of the scaffold 202 is exposed to the gas 212. Notably, a two-phase interface 216 is defined between the liquid 210 and the gas 212 and forms a meniscus. As the microfluidic device 200 is rotated, the meniscus/two-phase interface 216 will pass over a length of the scaffold 202. At 90° rotation, the meniscus/two-phase interface 216 is about half-way along a length of the scaffold 202. With 135° of rotation, the meniscus/two-phase interface 216 moves to the opposite side of the scaffold 202 from that shown at 45°, thus the meniscus/two-phase interface 216 is translating along an entire length of the scaffold such that a protein deposits and forms a matrix of suspended bridges of a plurality of extracellular matrix protein fibrils in the voids of the scaffold 202. As the fluidic device 200 further rotates back to the 0° position so that a full revolution has occurred, the meniscus/two-phase interface 216 has translated across an entire surface of the scaffold 202 to deposit protein therein. As will be appreciated by those of skill in the art, in addition to rotation, the fluidic device 200 may be tilted in other directions and/or be subject to other movements aside from rotation. The rotations of the fluidic device 200 may continue until the desired amount of extracellular matrix protein is deposited on or within voids of the scaffold 202.

FIG. 7 shows a fluidic (e.g., a microfluidic) device 230 prepared in accordance with certain other aspects of the present disclosure. Such a fluidic device 230 may be considered to be a tumbling design and used with a meniscus flow process described above. Alternatively, if the volume of liquid as compared to gas is increased, the fluidic device 230 may be used in a bubble flow process as described above. The device 230 includes a holding component 232 that may be a platform or substrate that has one or more regions 234 that may receive one or more three-dimensional polymeric scaffolds 236. The region(s) 234 of the holding component 232 may have dimensions that correspond to that of the three-dimensional scaffold 236 to be received therein, so that the scaffold 236 can be seated therein. The polymeric scaffolds 236 may be any of those described previously above, for example, a polycaprolactone (PCL) porous scaffold. In the design shown in FIG. 7, the holding component 232 may be a planar component that serves as a lid to seal an interior region 240. The interior region 240 is configured to receive liquid comprising a polymer and a gas. One or more ports 242 may be formed in a wall 244 of the fluidic device 230 for injecting a liquid comprising a protein and if necessary, additional gas into the interior region 240. One or more plugs 246 may be seated within the openings of the ports 242 to seal them when the fluidic device 230 is being used. The various components of the fluidic device 230, aside from the scaffolds 236, may be formed from a polymeric material, like polydimethylsiloxane (PDMS). In certain variations, the holding component 232 may be a glass layer having a layer of PDMS formed thereon that defines the regions 234 for receiving the scaffolds 236. In FIG. 7, two polymeric scaffolds 236 are disposed in the holding component 232, although the number, shape, dimensions, and the like are merely shown by way of example and are non-limiting. The fluidic device 230 may be used in a meniscus flow process like that shown in FIG. 6 to coat the scaffold(s) 236. Alternatively, the fluidic device 230 may be used in a bubble flow process to selectively coat the scaffold(s) 236 with deposited extracellular matrix protein.

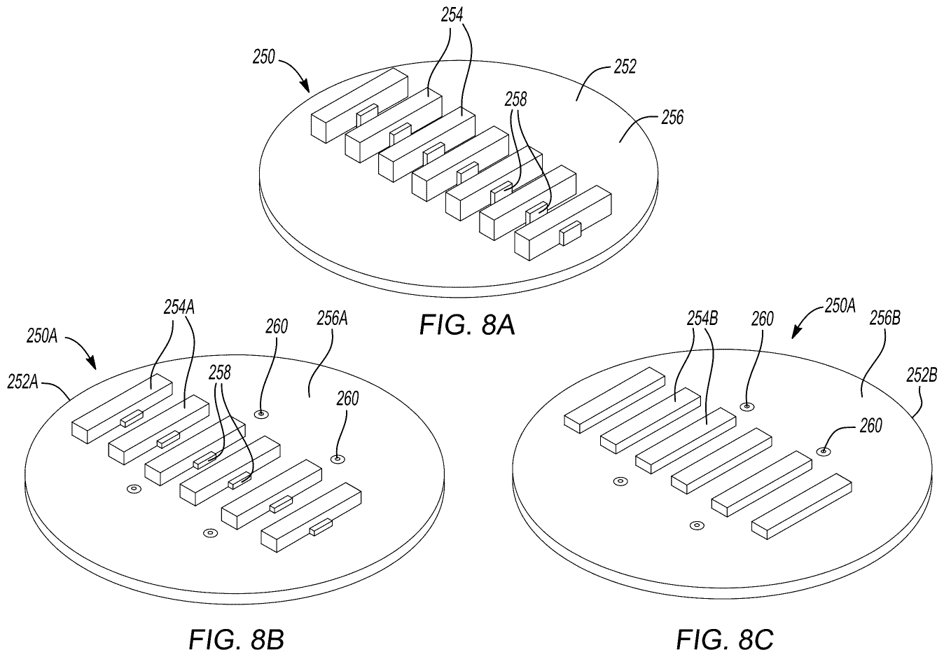

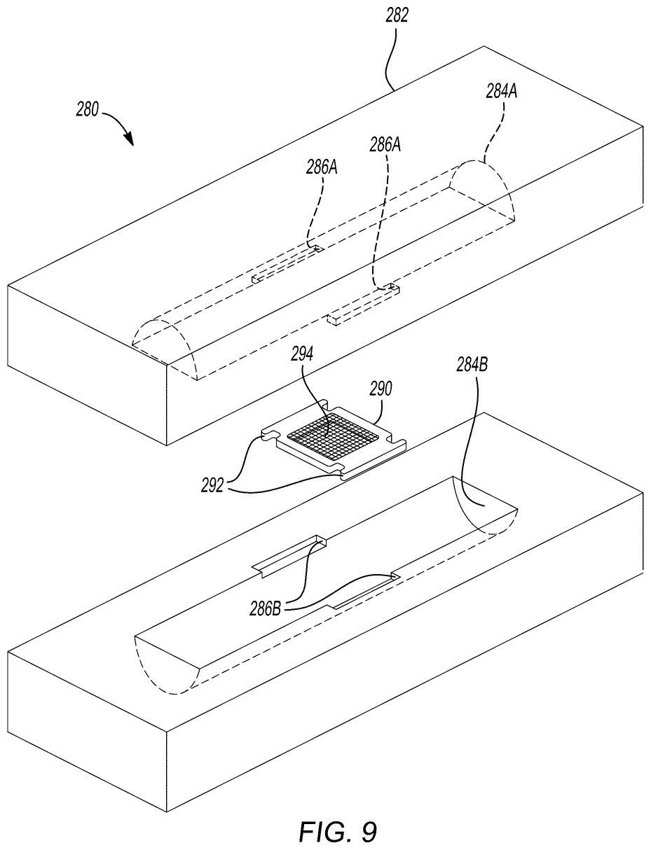

FIGS. 8A-8C show different variations of fluidic (e.g., a microfluidic) devices, specifically microfluidic device 250 in FIG. 8A and microfluidic device 250A in FIGS. 8B-8C prepared in accordance with certain other aspects of the present disclosure. Such fluidic devices 250, 250A may be considered to be a tumbling design. The fluidic devices 250, 250A may be used with a meniscus flow process described above. Alternatively, if the volume of liquid as compared to gas is increased, the fluidic devices 250, 250A may be used in a bubble flow process as described above. With respect to fluidic device 250, a two-part design is shown that has a lower component 252 that will be sealed by a planar lid or component (not shown). The lower component 252 has a plurality of channels 254 formed therein. The channels 254 are shown to have a rectangular shape with a rectangular cross-section, but are not limited to such shapes (as will be described in further detail below) or the number of channels shown 254. Notably, the plurality of channels 254 provides an array of microfluidic chambers so that multiple three-dimensional scaffold supports can be concurrently coated. The plurality of channels 254 may be molded into a round plate structure 256 and each molded channel 254 may have one or more pairs of lateral recesses 258 formed along each side of each channel 254. In certain aspects, the fluidic device 250 may be formed from polydimethylsiloxane (PDMS). The lateral recesses 258 may have a holder component (not shown) that retains a three-dimensional scaffold structure (not shown) on which the extracellular matrix protein will be coated to be seated therein during processing. The flat lid or plate will thus seal against the open side of the plurality of channels 254 after the three-dimensional scaffold(s) are disposed therein and form a fluid-tight seal for retaining liquid and/or gas disposed therein.

For the fluidic component 250A, another two-part design is shown that has a first lower component 252A that will be assembled together with a second upper component 252B. The first lower component 252A defines a first plurality of half-channels 254A, and the second upper component 252B defines a second plurality of half-channels 254B. Together, the first plurality of half-channels 254A and the second plurality of half-channels 254B will define a plurality of channels, such that the fluidic component 250A has a half-channel design for each of the respective parts. The first plurality of half-channels 254A and second plurality of half-channels 254B are shown to have a rectangular shape with a rectangular cross-section, but are not limited to such shapes (as will be described in further detail below) or the number of channels shown. In certain aspects, the fluidic device 250 may be formed from polydimethylsiloxane (PDMS). The first lower component 252A defines the first plurality of half-channels 254A that further may have one or more pairs of lateral recesses 258 formed along each side of each first half-channel 254A. The lateral recesses 258 may have a holder component (not shown) that retains a three-dimensional scaffold structure (not shown) on which the extracellular matrix protein will be coated to be seated therein during processing. The plurality of first half-channels 254A and second half-channels 254B may be molded into a round plate structures 256A, 256B and each molded channel 254 may have one or more pairs of lateral recesses 258 formed along each side of each channel 254. In certain aspects, the fluidic device 250A may be formed from polydimethylsiloxane (PDMS). When assembled together, the first lower component 252A and the second upper component 252B are fluid tight (and may further have sealing components or designs as known in the art). As shown, the first lower component 252A and the second upper component 252B may have one or more apertures 260 through which a mechanical fastener or/or adhesive may be received and secured.

FIG. 9 shows another variation of a two-part fluidic device 280 having a tumbling design where a first component 282 has a rectangular-shaped body with a first half-channel 284A formed therein. A second component 286 also has a rectangular-shaped body with a second half-channel 284B formed therein. When the first component 282 and the second component 286 are assembled, the first half-channel 284A and the second half-channel 284B together define a full cylindrical channel 284. The first plurality of half-channels 284A and second plurality of half-channels 284B are shown to have a circular or round shape with a cylindrical cross-section, but are not limited to such shapes (as will be described in further detail below) and may have more than a single channel, as shown. In certain aspects, the fluidic device 280 may be formed from polydimethylsiloxane (PDMS). The first component 282 may have one or more pairs of lateral recesses 286A formed along the sides of the first half-channel 284A. Likewise, the second component 286 may have one or more pairs of lateral recesses 286B formed along the sides of the second half-channel 284B. A holder component 290 may have two lateral flanges 292 that seat within the first lateral recesses 286A and second lateral recesses 286B when the first component 282 and the second component 286 are assembled together. The holder component 290 further comprises and retains a three-dimensional scaffold structure 294 on which the extracellular matrix protein will be coated to be seated therein during processing. When assembled together, the first component 282 and the second component 286 are fluid tight (and may further have sealing components or designs as known in the art).

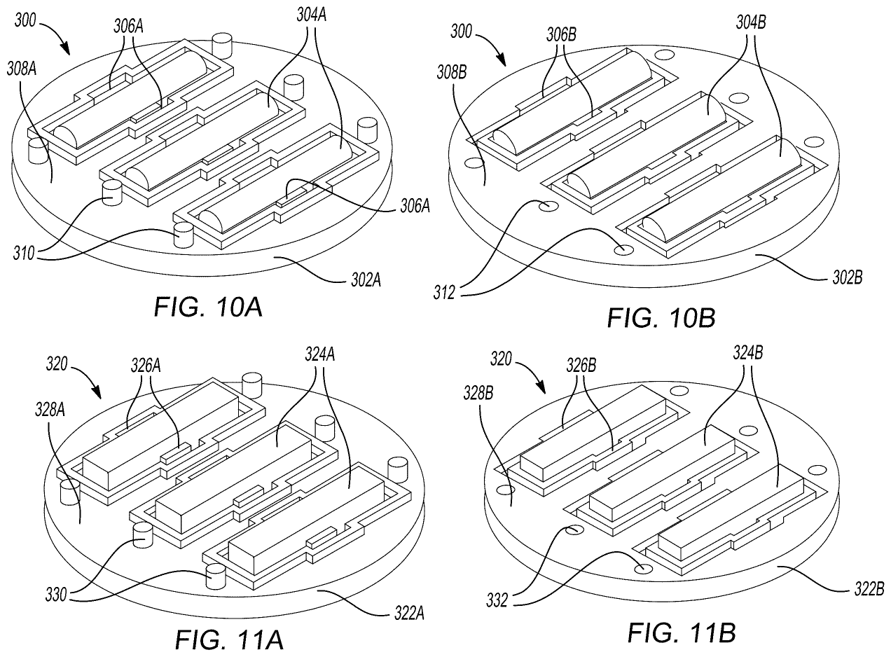

FIGS. 10A-10B show yet another variation of a fluidic (e.g., a microfluidic) device 300 prepared in accordance with certain other aspects of the present disclosure. Such a fluidic device 300 may be considered to be a tumbling design. The fluidic device 300 may be used with a meniscus flow process described above. Alternatively, if the volume of liquid as compared to gas is increased, the fluidic device 300 may be used in a bubble flow process as described above. The fluidic device 300 is yet another two-part design having a first component 302A and a second component 302B that will be assembled together. The first component 302A defines a first plurality of half-channels 304A, and the second component 302B defines a second plurality of half-channels 304B. Together, the first plurality of half-channels 304A and the second plurality of half-channels 304B will define a plurality of channels, such that the fluidic component 300 has a half-channel design for each of the respective parts. The first plurality of half-channels 304A and second plurality of half-channels 304B are shown to have a cylindrical shape with a circular or round cross-section, but are not limited to such shapes (as will be described in further detail below) or the number of channels shown. In certain variations, a cylindrical channel may be better suited for coating suspended three-dimensional scaffold structures, especially when using bubble flow methods of depositing fibronectin constructs. In certain aspects, the fluidic device 300 may be formed from polydimethylsiloxane (PDMS). The first plurality of half-channels 304A on the first component 302A may have one or more pairs of lateral recesses 306A formed along each side of each first half-channel 304A. The second plurality of half-channels 304B on the second component 302B may have one or more pairs of lateral recesses 306B formed along each side of each second half-channel 304B. The lateral recesses 306A, 306B may receive a holder component (not shown) that retains a three-dimensional scaffold structure (not shown) on which the extracellular matrix protein will be coated to be seated therein during processing. As shown in FIGS. 10A-10B, the first component 302A and the second component 302B have the plurality of first half-channels 304A and second half-channels 304B molded into round plate structures 308A and 308B. When assembled together, the first component 302A and the second component 302B are fluid tight (and may further have sealing components or designs as known in the art). As shown, the first component 302A has posts 310 protruding from the plate structure 302A, while the second component 302B may have one or more apertures 312 that receive the posts 310 and secure and seal the first component 302A to the second component 302B when they are assembled together.

FIGS. 11A-11B show yet another variation of a fluidic (e.g., a microfluidic) device 320 prepared in accordance with certain other aspects of the present disclosure. Such a fluidic device 320 may be considered to be a tumbling design. The fluidic device 320 may thus be used with a meniscus flow process described above. Alternatively, if the volume of liquid as compared to gas is increased, the fluidic device 320 may be used in a bubble flow process as described above. The fluidic device 320 is another two-part design having a first component 322A and a second component 322B that will be assembled together. The first component 322A defines a first plurality of half-channels 324A, and the second component 322B defines a second plurality of half-channels 324B. Together, the first plurality of half-channels 324A and the second plurality of half-channels 324B will define an array or plurality of channels, such that the fluidic component 320 has a half-channel design for each of the respective parts. The first plurality of half-channels 324A and second plurality of half-channels 324B are shown to have a rectangular shape with a rectangle or square cross-section, but the channels are not limited to such shapes or the number of channels shown. In certain aspects, the fluidic device 320 may be formed from polydimethylsiloxane (PDMS). The first plurality of half-channels 324A on the first component 322A may have one or more pairs of lateral recesses 326A formed along each side of each first half-channel 324A. The second plurality of half-channels 324B on the second component 322B may have one or more pairs of lateral recesses 326B formed along each side of each second half-channel 324B. The lateral recesses 326A, 326B may receive a holder component (not shown) that retains a three-dimensional scaffold structure (not shown) on which the extracellular matrix protein will be coated to be seated therein during processing. As shown in FIGS. 11A-11B, the first component 322A and the second component 322B have the plurality of first half-channels 324A and second half-channels 324B molded into round plate structures 328A and 328B. When assembled together, the first component 322A and the second component 322B are fluid tight (and may further have sealing components or designs as known in the art). As shown, the first component 322A has posts 330 protruding from the plate structure 322A, while the second component 322B may have one or more apertures 332 that receive the posts 330 and secure and seal the first component 322A to the second component 322B when they are assembled together.

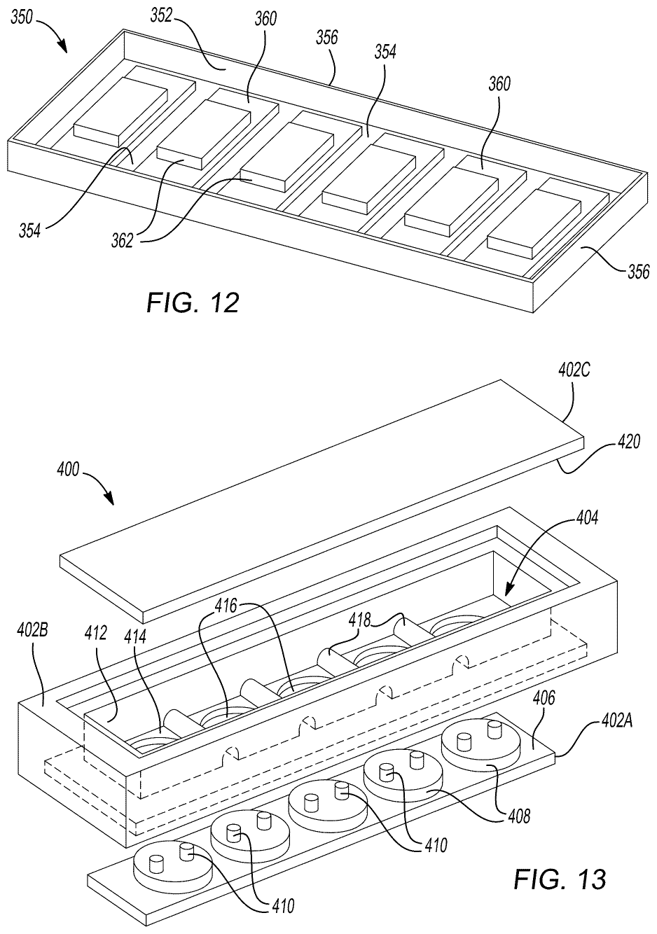

FIG. 12 shows a mold 350 for forming yet another fluidic (e.g., a microfluidic) device prepared in accordance with certain other aspects of the present disclosure. The mold 350 may be additive manufactured or three-dimensionally printed and then used to form a polymeric fluidic device, for example, comprising polydimethylsiloxane (PDMS). The fluidic device formed from mold 350 may be used with a meniscus flow process or a bubble flow process as described above. The fluidic device is yet another two-part design having a first component and a second component that will be assembled together. The mold 350 as shown may be used to form a first component of the assembly that will form the fluidic device. The mold for the second component is not shown in FIG. 12, but may simply be a planar or flat component, like a lid or slide that seats within the first component. The mold 350 may define an interior region 352 having a surface 354 surrounded by an exterior wall 356. A plurality of raised rectangular regions 360 each having a respective rectangular platform 362 are disposed on the surface 354. In this manner, the contrapositive shape to the mold 350 will define a plurality of open fluidic compartments (formed by the raised rectangular regions 360) having recesses (formed by the rectangular platforms 362) that may receive one or more three-dimensional polymeric scaffolds, as described previously above. For example, a polymer, such as PDMS may be poured into the mold 350 to achieve the rectangular chamber shape (e.g., where top rectangular platform 362 serves as a chamber space for fibronectin matrix and second rectangle (plurality of raised rectangular regions 360) serves as space that receives a second component, like a lid. The dimensions of plurality of raised rectangular regions 360 may correspond to and be fitted to the dimension of the second component (e.g., glass slide).

FIG. 13 shows yet another variation of a fluidic (e.g., a microfluidic) device 400 prepared in accordance with other aspects of the present disclosure. Such a fluidic device 400 is particularly suitable for use with a bubble flow process. The fluidic device 400 is a three-part assembly design that includes a first component 402A, a second component 402B, and a third component 402C that will be assembled together to form a fluid-tight internal chamber 404 that can receive liquid and gas for the processes of forming an extracellular matrix protein construct.

The first component 402A will form a lower region or bottom of the assembly (in the orientation shown) and has a planar surface 406 with a plurality of raised round stages 408. Each stage 408 has at least one pair of support posts 410 disposed thereon. As in previous embodiments, the support posts 410 can serve as initiation sites for creating a deposited protein construct (not shown) comprising a plurality of extracellular matrix protein fibrils as the bubble(s) passes over them. In this manner, the deposited protein construct can span between respective support posts 410. As noted above, the fluidic device 400 is particularly suitable for use with a bubble flow method, where the pillars or posts 410 serve as a contact point for the deposited protein (e.g., fibronectin matrix). The second component 402B defines a central opening 412 with a lower surface 414 having a plurality of round apertures 416 through which the plurality of stages 408 will be received when the first component 402A and second component 402B are assembled together. The lower surface 414 also comprises a plurality of long protrusions 418 that extend laterally from one side to the other of the central opening 412 and thus serve as barriers between each respective stage 408. The third component 402C may have a planar surface 420 that will serve as an upper surface or lid to seal the central opening 412 of the second component 402B. In this manner when the first component 402A, second component 402B, and third component 402C are assembled, the fluid-tight internal chamber 404 will contain liquid comprising the extracellular protein and a gas. As with previously embodiments, the various components in the fluidic device 400 may be formed of a polydimethylsiloxane (PDMS).

There are other device designs where an air-liquid interface can be utilized to scale-up the production of a fibronectin matrix in accordance with certain aspects of the present disclosure. Such designs allow a scaffold material to enter and exist at the air-liquid interface. In this manner, the fibronectin matrix will assemble into fibrillar structures across the scaffold material and these designs can effectively scale-up the production.

For example, in rotary printing, a scaffold material is placed on the rotary rod and is placed in contact with a protein solution. Rotation of the rod allows a scaffold material to travel across the air-liquid interface. A blade can be added to induce a more uniformly defined meniscus in x-, y-, and z-directions. In certain aspects, a blade can also be engineered to include microstructures to render increased versatility/functionality of controlling the enhanced flow and mixing of protein particles during the process. Rotary printing can be expanded to become “continuous” by incorporating a belt system, which would include a larger length of scaffold material to be fed on a continuous basis like a roll-to-roll/printing press. In another variation, such as a pressure/gravity driven flow system, controlled movement of the meniscus can be envisioned to assemble a fibronectin matrix across the surface of an object in its path. Pressure will give variable control in the speed, so that the meniscus is allowed to move.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A method of making a cellular support system comprising:
passing a bubble comprising gas through a liquid comprising an extracellular matrix protein contained in a fluidic device, wherein the bubble is passed over a target within the fluidic device to organize the extracellular matrix protein and form a deposited protein construct comprising a plurality of extracellular matrix protein fibrils on or near the target, wherein the deposited protein construct is capable of supporting cells and promotes three-dimensional cellular growth.

2. The method of claim 1, wherein the extracellular matrix protein is selected from the group consisting of: fibronectins, laminins, collagens, tenascins, elastin, vitronectin, periostin, and combinations thereof.

3. The method of claim 1, wherein the extracellular matrix protein comprises fibronectin.

4. The method of claim 1, wherein the fluidic device has a total volume and a volume fraction of the liquid to the total volume is greater than or equal to about 0.55.

5. The method of claim 1, wherein the plurality of extracellular matrix protein fibrils has an anisotropic orientation.

6. (canceled)

7. The method of claim 1, wherein the target comprises a three-dimensional scaffold structure comprising at least one void and the deposited protein construct spans over the at least one void and the three-dimensional scaffold structure is formed from a material selected from the group consisting of: a metal material, a polymeric material, a composite material, a ceramic material, a biologically derived material, and combinations thereof.

8. The method of claim 1, wherein the target comprises a three-dimensional scaffold structure comprising at least one void and the deposited protein construct spans over the at least one void and the three-dimensional scaffold structure comprises a polymer and is formed from a polymeric precursor or is a polymer selected from the group consisting of: polylactic acid, polyglycolide, polycaprolactone, poly(lactide-co-glycolide), poly(lactide-co-caprolactone), polyethylene glycol, starches, polydimethylsiloxane, polyurethanes, polyolefins, polyamides, celluloses, lignins, biodegradable polyesters, polystyrene, and combinations thereof.

9. The method of claim 1, wherein the target comprises at least one surface in the fluidic device such that the deposited protein construct coats the at least one surface.

10. The method of claim 1, wherein target comprises a first support and a second support, so that the passing of the bubble occurs from the first support to the second support and the deposited protein construct spans from the first support to the second support.

11. The method of claim 10, wherein the protein construct is removed from the first support and the second support for form a free-standing film.

12. The method of claim 1, wherein the passing the bubble comprises moving the fluidic device, wherein the moving is selected from the group consisting of: tipping, orbital rotation, tumbling rotation, spinning, vibration, shaking, and combinations thereof.

13. The method of claim 1, wherein the gas comprises air.

14. The method of claim 1, wherein the passing occurs for greater than or equal to about 5 minutes to less than or equal to about 96 hours.

15. The method of claim 1, wherein the liquid comprising extracellular matrix protein is a protein solution having a concentration of protein from about 0.01 mg/mL to about 2.5 mg/mL.

16. The method of claim 1, wherein the deposited protein construct occupies a surface area of greater than or equal to about 3.3 cm².

17. (canceled)

18. The method of claim 1, wherein the deposited protein construct has a dimension of greater than or equal to about 15 mm.

19. (canceled)

20. A fluidic device for forming a deposited protein construct, the fluidic device comprising:
a fluid-tight assembly comprising at least a first component and a second component that are assembled together to define an internal volume configured to contain a liquid comprising an extracellular matrix protein and a gas, wherein at least one of the first component or the second component comprises at least one target on which the extracellular matrix protein will organize and form a deposited extracellular matrix protein construct comprising a plurality of extracellular matrix protein fibrils on or near the at least one target, wherein the at least one target comprises: at least one pair of posts or at least one holding component configured to receive a three-dimensional scaffold support.

21. (canceled)

22. The fluidic device of claim 20, wherein the internal volume defines at least one elongated channel having a cross-sectional shape selected from the group consisting of: a rectangle, a square, a circle, an oval, and combinations thereof and either (i) the first component defines a first half of the at least one elongated channel and the second component defines a second half of the at least one elongated channel or (ii) the first component defines the at least one elongated channel and the second component defines a planar surface.

23. (canceled)

24. The fluidic device of claim 22 further comprising at least one pair of recessed regions adjacent to the at least one elongated channel configured to receive the at least one holding component configured to receive the three-dimensional scaffold support.

25-32. (canceled)

33. A method of expanding three-dimensional growth of cells comprising:
introducing cells to a cellular support comprising a deposited protein construct comprising a plurality of extracellular matrix protein fibrils aligned in an anisotropic orientation; and
growing the cells on the deposited protein construct in three-dimensions.

34-44. (canceled)
